(19) United States
(12) Patent Application Publication
(10) Pub. No.: US 2013/0226014 A1
(43) Pub. Date: Aug. 29, 2013

(54) SPHYGMOMANOMETER WITH ACCESSORY LOW-SPEED PRESSURE RELEASE DEVICE

(71) Applicant: Han-Chang Liu, New Taipei City (TW)

(72) Inventor: Han-Chang Liu, New Taipei City (TW)

(21) Appl. No.: 13/406,334

(22) Filed: Feb. 27, 2012

(51) Int. Cl. A61B 5/0235

(52) U.S. Cl. 600/498

(57) ABSTRACT

A sphygmomanometer with an accessory low-speed pressure release device comprises: a main body, having a multi-channel pipeline, and a pressure pump coupled to the multi-channel pipeline; a circuit device, including a microprocessor and a pressure sensor, and the pressure sensor is coupled to the multi-channel pipeline, and the circuit device is coupled to the pressure pump through an electric circuit; a cuff device, coupled to the multi-channel pipeline through an air channel; and an accessory low-speed pressure release device, installed on the air channel and disposed outside the main body, so that the accessory low-speed pressure release device is adjusted according to the cuffs of different sizes and operated at an optimal air release speed to obtain measurements more quickly and accurately when cuffs of different sizes are used.

### BACKGROUND OF THE INVENTION

1. Field of the Invention

This invention relates to an electronic sphygmomanometer, particularly the sphygmomanometer operated with a cuff with different sizes and using an external low-speed pressure release device and an optimal air release speed to obtain measurements more quickly and accurately when the cuff of a different size is used.

2. Description of Related Art

Sphygmomanometer is a common medical and healthcare measurement instrument, and the structure of a conventional sphygmomanometer 90 as shown in FIG. 1 comprises a main body 91 and a cuff device 96, and the main body 91 contains a multi-channel pipeline 93 installed therein and coupled to a pressure pump 911, an air release valve 912, a solenoid valve 913 and a pressure sensor 914 in different directions, wherein the pressure sensor 914 is installed on a circuit board 94 for sensing and measuring a blood pressure and a pulse from a user's arm, wherein high/low blood pressure values are measured, and the circuit board 94 includes a microprocessor (MCU) 941 installed thereon and coupled to a liquid crystal display 95 through an electric circuit, and the main body 91 is powered on by the control of a switch 915. In addition, the cuff device 96 includes a cuff 92 and a band 922, and the cuff 92 is coupled to the multi-channel pipeline 93 of the main body 91 through an air channel 921, and the band 922 is provided and wrapped around the user's arm.

In the aforementioned conventional sphygmomanometer 90, the solenoid valve 913 is provided for releasing the air in the cuff quickly, and the air release valve 912 is provided for releasing the air in the cuff at a constant air release speed by a low air flow during the process of measuring blood pressures. After the high/low blood pressure values measured by the pressure sensor 914 increase to a preset maximum pressure value when the cuff 92 is inflated and pressurized, the air in the cuff 92 is released constantly within a fixed time by a low air flow, so that the size of the cuff 92 will affect the time required for releasing the air during the measuring process. The cuff 92 also comes with small, standard, and large sizes to fit a user' arm with different sizes. In general, the small size is applicable for children and youngsters with an arm circumference less than 9 inches, the standard size is applicable for Asians or a general Europeans and Americans with an arm circumference of 9 to 13 inches, and the large size is applicable for obese people with an arm circumference of approximately 13 to 17 inches. Since the large cuff 92 comes with a large volume, it takes a longer pressure release time by using the air release valve at a constant pressure releasing speed, and also takes a longer time for users to measure their blood pressure, and the total measuring time taken is longer which will hold up the use by the next user. Obviously, the conventional sphygmomanometer requires improvements, and it is an important subject for designer and manufacturers of the related industry to overcome the drawbacks of the prior art.

In view of the aforementioned problems of the conventional sphygmomanometer, the inventor of the present invention based conducted extensive researches and experiments, and finally developed an electronic sphygmomanometer with an accessory low-speed pressure release device for measuring blood pressures more quickly and conveniently to overcome the drawbacks of the prior art.

### SUMMARY OF THE INVENTION

Therefore, it is a primary objective of the present invention to provide a sphygmomanometer with an accessory low-speed pressure release device adjustable according to different sized cuffs and operated at an optimal air release speed to achieve the effect of obtaining measurements more quickly and accurately when a different size cuff is used.

To achieve the aforementioned objectives, the present invention provides a sphygmomanometer, comprising: a main body, having a multi-channel pipeline, and a pressure pump coupled to the multi-channel pipeline; a circuit device, installed inside the main body, and including a microprocessor and a pressure sensor, and the pressure sensor being coupled to the multi-channel pipeline, and the circuit device being coupled to the pressure pump through an electric circuit; a cuff device, installed outside the main body, and including a cuff, and the cuff being coupled to the multi-channel pipeline through an air channel; and an accessory low-speed pressure release device, installed on the air channel and disposed outside the main body, and the accessory low-speed pressure release device including an accessory air release valve for releasing pressure at a low speed.

The technical characteristics and effects of the present invention will become apparent with the detailed description of preferred embodiments accompanied with related drawings as follows.

### BRIEF DESCRIPTION OF THE DRAWINGS

FIG. 1 is a schematic view of a conventional sphygmomanometer;

FIG. 2 is a schematic view of a preferred embodiment of the present invention;

FIG. 2a is a close-up view of an accessory low-speed pressure release device of the present invention;

FIG. 2b is a schematic view of FIG. 2a viewing from another angle; and

FIG. 3 is a schematic view of connecting a pipeline and a circuit in accordance with a preferred embodiment of the present invention.

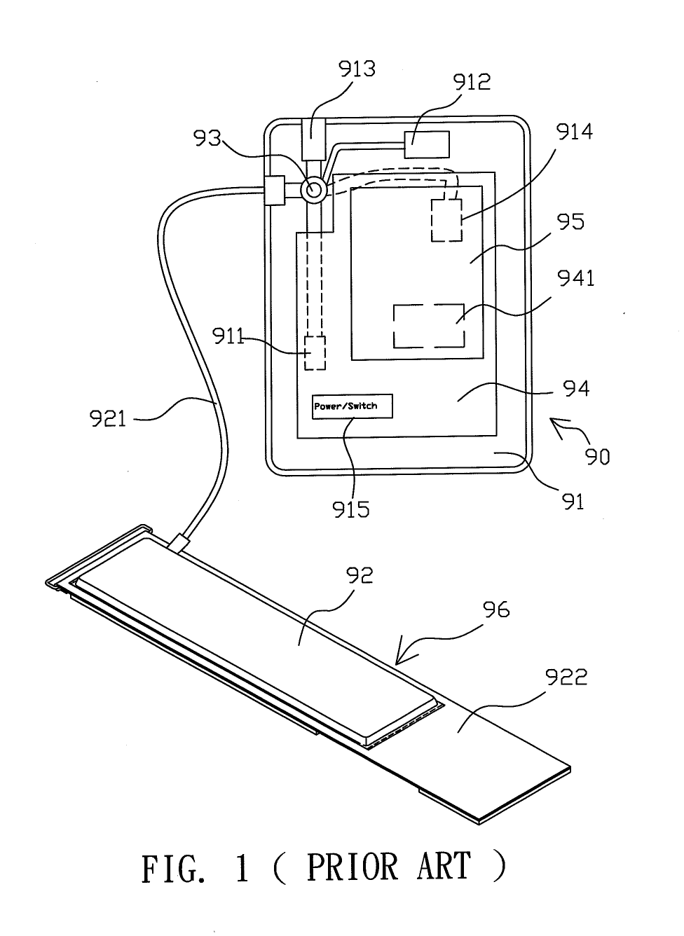

### DETAILED DESCRIPTION OF THE PREFERRED EMBODIMENTS

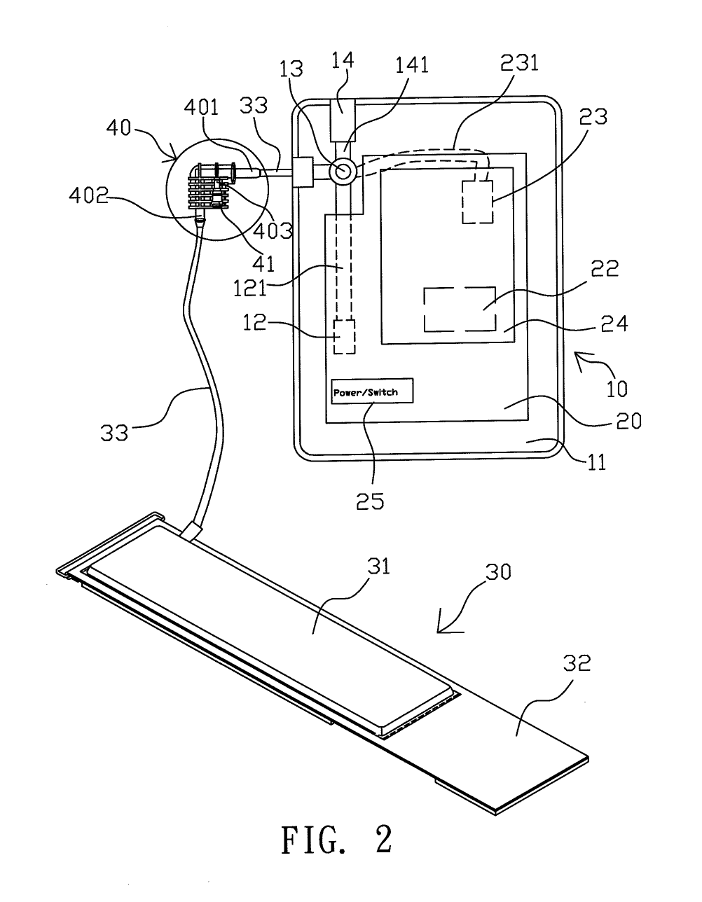

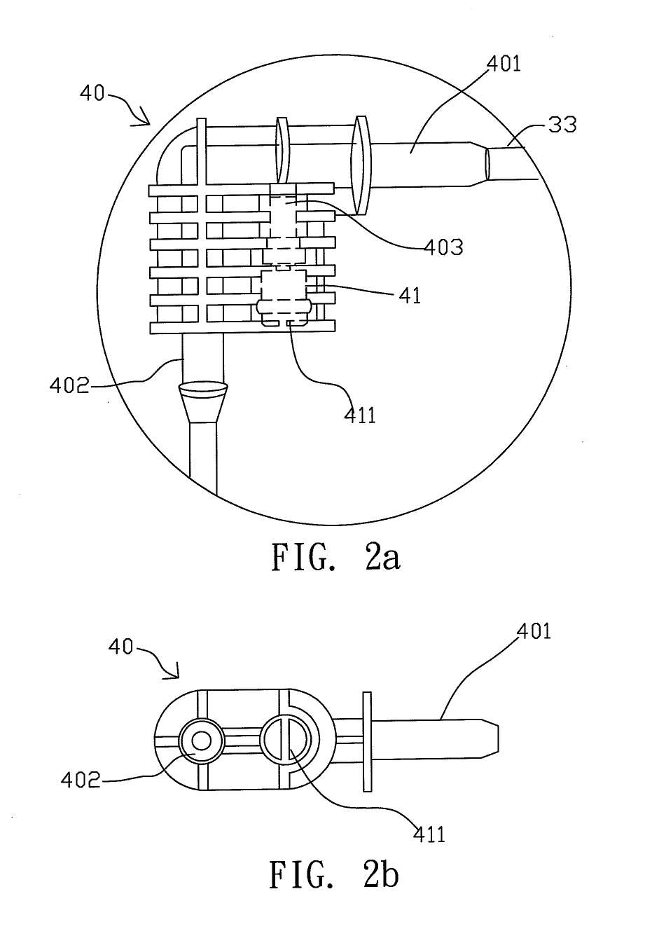

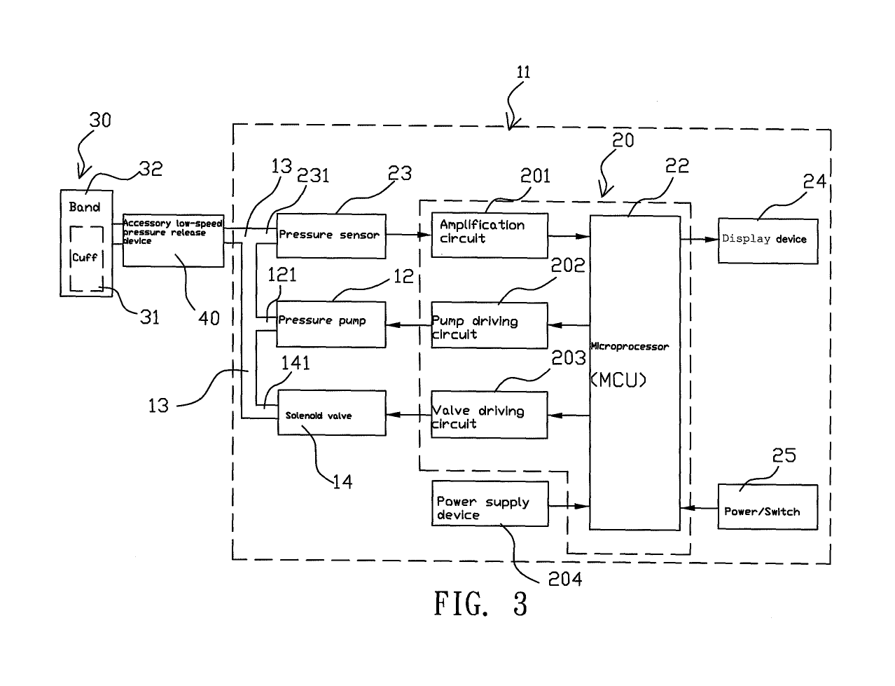

With reference to FIGS. 2 and 3 for a sphygmomanometer with an accessory low-speed pressure release device in accordance with a preferred embodiment of the present invention, the present invention comprises a sphygmomanometer 10, a circuit device 20, a cuff device 30 and an accessory low-speed pressure release device 40.

Wherein, the sphygmomanometer 10 comprises a main body 11, a multi-channel pipeline 13 installed in the main body 11 and coupled to a pressure pump 12 and a solenoid valve 14. In other words, the multi-channel pipeline 13 includes a pressurized pipe 121 for coupling the pressure pump 12 and a solenoid valve pipe 141 for coupling the solenoid valve 14; and the pressure pump 12 is provided for inflating and pressurizing the cuff device 30, and the solenoid valve 14 is provided for releasing the air in the cuff device 30 quickly.

Wherein, the circuit device 20 is installed in the main body 11, and the circuit device 20 of this preferred embodiment is a circuit board, and the circuit device 20 includes a microprocessor 22 (MCU) and a pressure sensor 23, and the pressure sensor 23 is provided for sensing and measuring a blood pressure and a pulse through a user's arm, and high/low blood pressure values are measured, and the pressure sensor 23 is coupled to the multi-channel pipeline 13 by a sensing pipe 231, and the circuit device 20 is coupled to a display device 24 and a switch for controlling the switch 25 of the power channel through an electric circuit.

Wherein, the cuff device 30 is installed outside the main body 11 and includes a cuff 31 and a band 32 coupled with the cuffs 31 and covered around the user's arm, and the cuff 31 is coupled to the multi-channel pipeline 13 of the main body 11 through an air channel 33.

Wherein, the accessory low-speed pressure release device 40 as shown in FIGS. 2, 2a and 2b is installed on the air channel 33 and disposed outside the main body 11. The accessory low-speed pressure release device 40 includes an accessory air release valve 41, and the accessory low-speed pressure release device 40 has a multi-channel connecting channel, and the multi-channel connecting channel includes a plurality of connecting channels 401, 402 for connecting the air channels 33 at both ends respectively, and a connecting channel 403 for connecting (or installing) the accessory air release valve 41. In practical applications, the connecting channels 401, 402 of the accessory low-speed pressure release device 40 can be coupled directly to the cuff device 30 or the multi-channel pipeline 13 of the main body 11. The accessory air release valve 41 is provided for assisting a low-speed pressure release of the main body 11 and includes an adjusting button 411 for facilitating an adjustment of the air release quantity of the accessory low-speed pressure release device 40 while adjusting the air release according to different sized cuffs 31.

In FIG. 3, the circuit device 20 further includes an amplification circuit 201, a pump driving circuit 202 and a valve driving circuit 203 electrically coupled to the microprocessor 22 (MCU) and used for receiving and amplifying a signal of the pressure sensor 23 and driving the pressure pump 12 and the solenoid valve 14 respectively. The microprocessor 22 (or the circuit device 20) is coupled to a power supply device 204 for supplying electric power required by the operation of the present invention, and transmitting related information to the display device 24 for display.

The present invention adjusts the accessory low-speed pressure release device 40 installed on the air channel 33 outside the main body 11 according to different sized cuffs 31 and operates the accessory low-speed pressure release device at an optimal air release speed to obtain measurements more quickly and accurately when different sized cuffs 31 are used.

With the aforementioned components and structure, the sphygmomanometer with the accessory low-speed pressure release device according to the present invention can adjust the accessory low-speed pressure release device according to the different sized cuffs and operate it at the optimal air release speed to achieve the effect of obtaining measurements more quickly and accurately when different sized cuffs are used.

In summation of the description above, the present invention overcomes the problems of the prior art and complies with patent application requirements, and is thus duly filed for patent application. While the invention has been described by means of specific embodiments, numerous modifications and variations could be made thereto by those skilled in the art without departing from the scope and spirit of the invention set forth in the claims.

What is claimed is:

1. A sphygmomanometer with an accessory low-speed pressure release device, comprising:
a main body, having a multi-channel pipeline, and a pressure pump coupled to the multi-channel pipeline;
a circuit device, installed inside the main body, and including a microprocessor and a pressure sensor, and the pressure sensor being coupled to the multi-channel pipeline, and the circuit device being coupled to the pressure pump through an electric circuit;
a cuff device, installed outside the main body, and including a cuff, and the cuff being coupled to the multi-channel pipeline through an air channel; and
an accessory low-speed pressure release device, installed on the air channel and disposed outside the main body, and the accessory low-speed pressure release device including an accessory air release valve for releasing pressure at a low speed.

2. The sphygmomanometer with an accessory low-speed pressure release device according to claim 1, wherein the multi-channel pipeline has a solenoid valve coupled thereto.

3. The sphygmomanometer with an accessory low-speed pressure release device according to claim 1, wherein the circuit device is coupled to a liquid crystal display through an electric circuit.

4. The sphygmomanometer with an accessory low-speed pressure release device according to claim 1, wherein the cuff device includes a band combined with to the cuff.

5. The sphygmomanometer with an accessory low-speed pressure release device according to claim 1, wherein the accessory low-speed pressure release device comprises a plurality of connecting channels including a connecting channel for connecting the air channel and a connecting channel for connecting the accessory air release valve.

6. The sphygmomanometer with an accessory low-speed pressure release device according to claim 1, wherein the circuit device further comprises an amplification circuit, a pump driving circuit and a valve driving circuit, and the circuit device is electrically coupled to a power supply device.

7. The sphygmomanometer with an accessory low-speed pressure release device according to claim 1, wherein the accessory air release valve includes an adjusting button.

8. The sphygmomanometer with an accessory low-speed pressure release device according to claim 1, wherein the accessory air release valve device has a connecting channel coupled to both ends of the accessory air release valve device, and coupled and provided for sheathing on the air channel.

9. The sphygmomanometer with an accessory low-speed pressure release device according to claim 1, wherein the accessory air release valve device has an end sheathed on the air channel, and the other end coupled to the multi-channel pipeline directly.

10. The sphygmomanometer with an accessory low-speed pressure release device according to claim 1, wherein the accessory air release valve device has an end sheathed on the air channel and the other end coupled to the cuff device.
